(19) United States
(12) Patent Application Publication
(10) Pub. No.: US 2020/0138823 A1
(43) Pub. Date: May 7, 2020

(54) DRUG COMPOSITIONS

(71) Applicant: Adamis Pharmaceuticals Corporation, San Diego, CA (US)

(72) Inventors: David Clayton Sutherland, Little Rock, AR (US); James Kyle Zorn, Lexington, KY (US); Dennis J. Carlo, San Diego, CA (US)

(21) Appl. No.: 16/672,323

(22) Filed: Nov. 1, 2019

Related U.S. Application Data
(63) Continuation of application No. 62/770,599, filed on Nov. 21, 2018.
(60) Provisional application No. 62/754,966, filed on Nov. 2, 2018.

(51) Int. Cl. A61K 31/53; A61K 31/4184; A61K 9/06; A61K 47/10; A61K 9/00; A61K 31/47; A61P 33/02

(52) U.S. Cl. CPC A61K 31/53; A61K 31/4184; A61K 9/06; A61P 33/02; A61K 9/0053; A61K 31/47; A61K 47/10

(57) ABSTRACT

Described herein are pharmaceutical compositions including in combination at least one antiparasitic drug and one antiprotozoal drug. In some embodiments, the compositions can be formulated as a non-solid for oral administration. The compositions can be used to treat neurological conditions. Methods of treatment using the compositions are also described.

### CROSS REFERENCE TO RELATED APPLICATIONS

This application claims priority to United States Provisional Patent Application Ser. No. 62/754,966, filed Nov. 2, 2018, and 62/770,599, filed Nov. 21, 2018, each of which are incorporated by reference herein in their entireties.

### FIELD

Described herein are compositions including combination therapies for treating neurological conditions.

### SUMMARY

Described herein are pharmaceutical compositions useful for treating and/or preventing neurological diseases in mammals. “Mammals” can include, but are not limited to humans, horses, camels, dogs, cats, cows, bears, rodents, sheep, goats, pigs and the like. In some embodiments, the compositions described herein can be considered veterinary compositions. In some embodiments, the compositions can include a synergistic combination of drugs and/or have an additive drug effect and can be termed combination therapy. In some embodiments, the combination therapy can include an antiprotozoal drug and an antiparasitic drug.

In one embodiment, the antiprotozoal drug is toltrazuril or decoquinate and the antiparasitic drug is fenbendazole. In some embodiments, the antiprotozoal drug and antiparasitic drug are is present at a concentration of, for example, about 20-30% w/v each.

Combining toltrazuril or decoquinate with fenbendazole can provide a surprising synergistic effect that can reduce neurological recovery time and/or reduce the amount of drugs needed to achieve a similar or better result when compared to a single drug.

In some embodiments, the composition is formulated as a non-solid, for example for oral administration. The non-solid can be, for example, a liquid or a paste.

The compositions can further include polyethylene glycol.

Methods of treating neurological conditions such as Equine Protozoal Myeloencephalitis (EPM) are also described. The methods can comprise administering a composition including the antiprotozoal drug and the antiparasitic drug to a mammal having the neurological condition.

In some embodiments, the administration is performed using an oral syringe.

In some embodiments, the mammal is a human, a horse, or a camel. In some embodiments, the mammal is an athlete.

Methods of forming a composition including an antiprotozoal drug and antiparasitic drug are also described. The methods can comprise mixing a combination of the antiprotozoal drug and antiparasitic drug in a melted base agent to form the composition.

In some embodiments, the melted base agent is polyethylene glycol. The mixed composition can also optionally include a preservative and/or a wetting agent.

### DETAILED DESCRIPTION

Prior to development of the present compositions, neurological conditions such as EPM remained difficult to diagnose and treat. For example, because not all horses exposed to the protozoa responsible develop the disease, the presence of antibodies is not enough to diagnose EPM. In addition, even with antiprotozoal drugs, the recovery rate is about 65 percent. Historically, treatment options included:
Ponazuril (tradename Marquis; generic name toltrazuril sulfone), an oral paste administered once daily for 28 days.
Pyrimethamine and sulfadiazine (tradename Rebalance), an oral suspension administered once daily for as long as 120 days.
Diclazuril (tradename Protazil), a pelleted, alfalfa-based top-dressing fed for 28 days.

All of these treatments have similar efficacy.

Described herein are pharmaceutical compositions and formulations useful for treating neurological conditions in mammals, for example conditions of the brain, spine, and nervous system. Neurological conditions can include, for example, Equine Herpesvirus type 1 (EHV-1) Equine Protozoal Myeloencephalitis (EPM), West Nile virus, Cervical stenotic myelopathy (CVM; “Wobbler Syndrome”), combinations thereof, and the like. In some embodiments, the herein-described pharmaceutical compositions can be used to treat and/or prevent EPM. In embodiments, treating can comprise a lessening of symptoms in the mammal.

Disclosed drug combinations can produce synergistic and/or additive effects in reducing or preventing a symptom associated with a neurological condition. Consequently, a considerably reduced dose of therapeutic compounds can be given for an equivalent effect for each individual therapeutic compound or an equivalent amount of each therapeutic compound can be given to achieve a larger and/or more rapid response. In some embodiments, the compositions can reduce side-effects and drug burden.

“Mammals” can include, but are not limited to, humans, horses, camels, dogs, cats, cows, bears, rodents, oxen, bison, buffalo, caribou, moose, deer, elk, sheep, goats, pigs, rabbits, pouched mammals, primates, carnivores, and the like.

In certain embodiments, the mammals can be athletes. Athletes can include, but are not limited to, race horses, race camels, race dogs, racing humans, jumping horses, jumping camels, jumping dogs, jumping humans, dancing horses, dancing camels, dancing dogs, dancing humans, and the like. In other embodiments, the mammals can be working mammals. Working mammals can include, but are not limited to, mammals that exert physical energy for a purpose. That purpose can be pulling a load, pushing a load, carrying a load, carrying a human, jumping, dancing, climbing, swimming, and the like.

In some embodiments, the mammal is a thoroughbred horse. Thoroughbreds can include, but are not limited to Arabians, Standardbreds, Quarter Horses, and the like.

As used herein, the term “pharmaceutical composition” refers to a therapeutically effective concentration of the drug or drugs and other ingredients described herein. As used herein, the term “pharmaceutically acceptable” refers to compositions that do not produce an adverse, allergic, or other untoward or unwanted reaction when administered to a mammal.

The drug combinations or combination therapies disclosed herein can be formulated in any appropriate form. An appropriate from can be one that is easy to administer, provides for the stability of active agent(s), allows the introduction of active agent(s), and the like. Appropriate forms can include, for example, powders, semi-solids, liquids, pastes, suspensions, inhalable dry powders, inhalable formulations, solids, granules, combinations thereof, and the like.

The drug combinations disclosed herein can be made into various formulations, for example a semi-solid formulation. Semi-solid formulations can be made for enteral or topical administration. Semi-solid formulations suitable for enteral administration include, without limitation, pastes, and gels. Semi-solid formulations suitable for topical or oral administration include, without limitation, ointments, creams, salves, pastes, and gels. The drug combination or combination therapy disclosed herein intended for such administration may be prepared according to any method known in the art for the manufacture of pharmaceutical compositions.

The drug combinations may be made into a liquid formulation. Liquid formulations suitable for enteral or parenteral administration include, without limitation, solutions, syrups, elixirs, dispersions, emulsions, and suspensions. The drug combination or combination therapy disclosed herein intended for such administration may be prepared according to any method known in the art for the manufacture of pharmaceutical compositions. In such liquid dosage forms, the drug combinations disclosed herein may be admixed with (a) suitable aqueous and non-aqueous carriers, (b) diluents, (c) solvents, such as, e.g., water, ethanol, propylene glycol, polyethylene glycol, glycerol, vegetable oils, such as, e.g., rapeseed oil and olive oil, and injectable organic esters such as ethyl oleate; and/or fluidity agents, such as, for example, surfactants or coating agents like lecithin. In the case of dispersions and suspensions, fluidity can also be controlled by maintaining a particular particle size.

Liquid and/or semi-solid formulations may be formulated with sweetening agents, for example glycerol, propylene glycol, sorbitol or sucrose. Such formulations may also contain one or more demulcent, preservative, flavoring agent, and/or coloring agent.

Liquid and/or semi-solid suspensions may be formulated by suspending the drug combination in an admixture with suitable excipients. Suitable excipients can be suspending agents, such as sodium carboxymethyl cellulose, methylcellulose, hydroxypropylmethylcellulose, sodium alginate, pectin, polyvinyl pyrrolidone, polyvinyl alcohol, natural gum, agar, gum tragacanth, and/or gum acacia.

Oily suspensions may be formulated by suspending the drug combination in an admixture with (a) vegetable oils including almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof, such as palmitic acid, stearic acid, oleic acid, linoleic acid, linolenic acid, or a combination thereof, (c) mineral oil such as liquid paraffin, (d) surfactants or detergents. The oily suspensions may contain a thickening agent such as beeswax, hard paraffin, or cetyl alcohol.

In some embodiments, oils can be used as a carrier or in the place of a carrier such as polyethylene glycol. The drug combinations disclosed herein may be made into an inhaled formulation. Inhaled formulations suitable for enteral or parenteral administration include, without limitation, aerosols and/or dry powders. The drug combination disclosed herein intended for such administration may be prepared according to any method known in the art for the inhalable manufacture of pharmaceutical compositions.

The drug combination may be made into a solid formulation. Solid formulations suitable for enteral or parenteral administration include, without limitation, capsules, tablets, pills, troches, lozenges, powders and granules suitable for inhalation, ingestion, or for reconstitution into sterile injectable solutions or dispersions. In other embodiments, the drug combination can be in a semi-solid form such as a paste. The drug combination intended for such administration may be prepared according to any method known to the art for the manufacture of pharmaceutical compositions. In such solid dosage forms, the drug combination may be admixed with (a) at least one inert customary excipient (or carrier), such as, e.g., sodium citrate or dicalcium phosphate or (b) fillers or extenders, such as for example, starch, lactose, sucrose, glucose, mannitol, isomalt, and silicic acid, (c) binders, such as, e.g., carboxymethylcellulose, alginates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated or they may be coated by known techniques to delay disintegration and absorption in the gastrointestinal tract and thereby provide a sustained action over a longer period.

In some embodiments, the compositions can include a combination of at least one antiparasitic drug and at least one antiprotozoal drug. In some embodiments, each of the antiparasitic and antiprotozoal drugs can be included in the composition at most about 35% (w/v), at most about 40% (w/v), about 15% (w/v), about 20% (w/v), about 25% (w/v), about 30% (w/v), about 35% (w/v), about 40% (w/v), about 45% (w/v), about 50% (w/v), between about 20% and about 40% (w/v), or between about 25% and about 35% (w/v). In some embodiments, each antiparasitic drug can be included in the composition at most about 30% (w/v), at most about 25% (w/v), about 10% (w/v), about 15% (w/v), about 20% (w/v), about 21% (w/v), about 22% (w/v), about 23% (w/v), about 25% (w/v), about 30% (w/v), about 35% (w/v), about 40% (w/v), between about 20% and about 25% (w/v), or between about 15% and about 30% (w/v). In embodiments, the antiparasitic and antiprotozoal drugs are not present in the same amount or percentage.

In some embodiments, each of the antiparasitic and antiprotozoal drugs in combination can be included in the composition at most about 30% (w/v), at most about 25% (w/v), about 10% (w/v), about 15% (w/v), about 20% (w/v), about 21% (w/v), about 22% (w/v), about 23% (w/v), about 25% (w/v), about 30% (w/v), about 35% (w/v), about 40% (w/v), between about 20% and about 25% (w/v), or between about 15% and about 30% (w/v).

In embodiments, disclosed compositions can comprise an antiparasitic and an antiprotozoal drug, for example at a 50:50 ratio. Further embodiments comprise an antiparasitic and an antiprotozoal drug, with the antiprotozoal drug, for example toltrazuril or decoquinate, at a ratio 25:20 as compared to the antiparasitic drug. Further ratios reflecting the amount of the antiprotozoal drug as compared to the antiparasitic drug can be, for example, 4:1, 3:1, 2:1, 1.5:1, 1.25:1, 1:1, 0.75:1, 0.5:1, 0.25:1, or the like. Further embodiments comprise an antiparasitic and an antiprotozoal drug, with the antiparasitic drug, for example fenbendazole, at a ratio 20:25 as compared to the antiprotozoal drug. Further ratios reflecting the amount of the antiparasitic drug as compared to the antiprotozoal drug can be, for example, 1:4, 1:3, 1:2, 1:1.5, 1:1.25, 1:1, 1:0.75, 1:0.5, 1:0.25, or the like.

In one embodiment, at least one of the antiparasitic drugs is fenbendazole. In one embodiment, at least one of the antiprotozoal drugs is toltrazuril. In one embodiment, at least one of the antiprotozoal drugs is decoquinate.

Combining toltrazuril with fenbendazole can provide a surprising synergistic effect that can reduce EPM recovery time. In other embodiments, combining toltrazuril or decoquinate with fenbendazole can provide a surprising synergistic effect that can reduce recovery time for other neurological conditions.

A composition disclosed herein may optionally include a pharmaceutically-acceptable carrier that facilitates processing of a drug into pharmaceutically-acceptable compositions. Such a carrier can be mixed with a drug or drugs or permitted to dilute or enclose the drug or drugs and can be a solid, semi-solid, or liquid agent. It is understood that the drug or drugs can be soluble or can be delivered as a suspension in the desired carrier or diluent. In some embodiments, the drugs can be formulated as a paste. Any of a variety of pharmaceutically acceptable carriers can be used including, without limitation, aqueous media such as, for example, water, saline, glycine, hyaluronic acid and the like; solid carriers such as, for example, polyethylene glycol, polyethylene oxide, mannitol, lactose, starch, magnesium stearate, sodium saccharin, talcum, cellulose, glucose, sucrose, magnesium carbonate, and the like; solvents; dispersion media; coatings; isotonic and absorption delaying agents; or any other inactive ingredient. Selection of a pharmacologically acceptable carrier can depend on the mode of administration. In some embodiments, solid carriers can be melted.

In one embodiment, the pharmaceutically-acceptable carrier is a base agent melted to form a paste. In some embodiments, each base agent can be included in the composition at most about 10% (w/v), at most about 6% (w/v), about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), between about 1% and about 10% (w/v), or between about 5% and about 7% (w/v).

In one embodiment, the base agent can be a polyethylene glycol. Polyethylene glycol used as a base agent can have a molecular weight of greater than about 1,000 g/mol, greater than about 1,200 g/mol, greater than about 1,400 g/mol, between about 1,000 g/mol and about 1,600 g/mol, between about 1,300 g/mol and about 1,500 g/mol, or between about 1,400 g/mol and about 1,500 g/mol. In one embodiment, the polyethylene glycol has a molecular weight of about 1,450 g/mol.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, flavoring agents, coloring agents, suspension agents, and the like.

Pharmaceutically acceptable antioxidants can include, without limitation, sodium metabisulfite, sodium thiosulfate, acetylcysteine, butylated hydroxyanisole and butylated hydroxytoluene.

Flavoring agents can provide a composition that smells good and/or tastes good. Non-human mammals may need encouragement to consume the compositions described. Thus, flavoring agents can be added that stimulate appeal to or that naturally attract a particular mammal species. Flavoring agents can make orally administrable compositions taste like apple, orange, lemon, grape, butterscotch, cherry, blueberry, raspberry, strawberry, honey, peppermint, spearmint, cinnamon, peach, watermelon, chocolate, espresso, mango, banana, carrot, cantaloupe, guava, acai, cheese, tomato, caramel, taffy, lime, and the like. Flavor combinations can also be provided.

Tonicity adjustors suitable for use with disclosed compositions can include, without limitation, salts such as, for example, sodium chloride, potassium chloride, mannitol or glycerin and other pharmaceutically acceptable tonicity adjustors.

Suspension agents can include, without limitation, carboxymethyl cellulose. The suspension agent can be used to keep the drug(s) or active agent dispersed evenly in the composition.

In some embodiments, the suspension agent(s) can be included in the composition at most about 0.2% (w/v), at most about 0.5% (w/v), about 0.05% (w/v), about 0.1% (w/v), about 0.2% (w/v), about 0.3% (w/v), about 0.4% (w/v), about 0.5% (w/v), about 0.8% (w/v), about 1% (w/v), between about 0.1% and about 0.3% (w/v), or between about 0.1% and about 1% (w/v).

Wetting agents can include, without limitation, polyethylene glycol. In some embodiments, the polyethylene glycol can be a low molecular weight polyethylene glycol. The low molecular weight polyethylene glycol can have a molecular weight of less than about 500 g/mol, less than about 300 g/mol, greater than about 100 g/mol, between about 200 g/mol and about 400 g/mol, between about 100 g/mol and about 500 g/mol, or between about 250 g/mol and about 350 g/mol. In one embodiment, the polyethylene glycol has a molecular weight of about 300 g/mol.

In some embodiments, the wetting agent can be included in the composition at least about 35% (w/v), at least about 60% (w/v), about 35% (w/v), about 40% (w/v), about 50% (w/v), about 60% (w/v), about 65% (w/v), about 70% (w/v), between about 35% and about 40% (w/v), or between about 60% and about 70% (w/v).

In one embodiment, a composition can include drugs formulated as a paste including polyethylene glycol. In some embodiments, a composition can include drugs formulated as a liquid including polyethylene glycol. In one embodiment, a composition can include toltrazuril, fenbendazole, and polyethylene glycol in solution. In one embodiment, a composition can include decoquinate, fenbendazole, and polyethylene glycol in solution.

In some embodiments, a composition can be provided as granules. In some embodiments, the granules can be provided as a scoop. A scoop can be provided as a 30 Scoop, a 60 Scoop, or a 100 Scoop size.

In some embodiments, a composition can be provided as a suspension. A suspension can be provided as, for example, a 480 mL aliquot, a 960 mL aliquot, or a 3,785 mL aliquot.

In one embodiment, a composition or combination therapy can include about 2.5 g toltrazuril, about 2.0 g fenbendazole, and about 1.2 g polyethylene glycol in 10 mL of solution.

In one embodiment, a composition or combination therapy can include about 2.5 g decoquinate, about 2.0 g fenbendazole, and about 1.2 g polyethylene glycol in 10 mL of solution.

In one embodiment, a composition or therapy can include a disclosed composition in a tube comprising 25 g toltrazuril, 20 g fenbendazole, and a pharmaceutically acceptable carrier.

In one embodiment, a composition or therapy can include a disclosed composition in a tube comprising 25 g decoquinate, 20 g fenbendazole, and a pharmaceutically acceptable carrier.

The composition can be stored at a temperature between about 20° C. and about 25° C.

In some embodiments, the composition can be provided in a dial-a-dose oral syringe. In one embodiment, the dial-a-dose syringe is a 80 mL syringe.

In some embodiments, the compositions can include inactive ingredients such as, but not limited to, polyethylene glycol, distilled water, and optionally sodium hydroxide. In other embodiments, each 1 mL of composition can include inactive ingredients such as, but not limited to, 2 mg of carboxymethylcellulose, 3.8 mg of sodium bicarbonate, 1 mg of sodium sulfite, 60 mg of polyethylene glycol 1450, 536 mg of polyethylene glycol 3, distilled water, and optionally sodium hydroxide to adjust pH.

The drug combination or combination therapy disclosed herein may be formulated for either local or systemic delivery using topical, enteral, or parenteral routes of administration.

In some embodiments, the compositions described herein are delivered orally. When delivered orally, the compositions can be administered using an oral medication syringe.

The compositions described herein can be administered at amounts of about 10 cc, about 15 cc, about 20 cc, about 25 cc, about 30 cc, about 35 cc, about 40 cc, about 45 cc, about 50 cc, about 55 cc, about 60 cc, about 65 cc, about 70 cc, about 75 cc, about 80 cc, about 85 cc, about 90 cc, about 95 cc, about 100 cc, at least about 10 cc, at least about 20 cc, at least about 25 cc, at least about 30 cc, at least about 35 cc, at least about 40 cc, at least about 45 cc, at least about 50 cc, at least about 60 cc, between about 10 cc and about 100 cc, between about 20 cc and about 80 cc, between about 40 cc and about 80 cc, between about 20 cc and about 30 cc, between about 25 cc and about 35 cc, between about 30 cc and about 35 cc, or between about 25 cc and about 30 cc per administration.

Administration can be any interval that results in a therapeutic response. Disclosed methods can comprise use of a loading dose, an initial higher dose of a drug that may be given at the beginning of a course of treatment before dropping down to a lower maintenance dose.

In some embodiments, administration can be one, two, three, four, five, or more times per day. In other embodiments, administration can be every other day, every third day, every fourth day, every fifth day, every sixth day, once per week, twice per month, monthly, and the like. In one embodiment, administration is once per day. In still other embodiments, administration can be for the remaining life of the mammal.

In some embodiments, when the animal is an athlete, administration can be before an athletic event such, as for example, but not limited to every day, every other day, every third day, every fourth day, every fifth day, every sixth day, once per week, twice per month, and the like leading up to the athletic event.

In other embodiments, when the animal is an athlete, administration can be after an athletic event such as, for example, but not limited to every day, every other day, every third day, every fourth day, every fifth day, every sixth day, once per week, twice per month, and the like after the athletic event.

In some embodiments, administration can include both before and after an athletic event. In some embodiments, the administration is the same before and after an athletic event. In other embodiments, the administration is different before and after the athletic event.

Methods of forming administrable compositions are also described. Methods can include first weighing the ingredients for the composition. Then, the polyethylene glycol base agent is heated until it melts. To the melted base agent, toltrazuril, and fenbendazole are added. The contents are then mixed until a smooth paste is achieved. The paste is wetted as necessary with a polyethylene glycol wetting agent. The resulting paste is then pulled into a syringe for storage, sterilization, administration, or the like. In another embodiment, to the melted base agent, decoquinate, and fenbendazole are added. The contents are then mixed until a smooth paste is achieved. The paste is wetted as necessary with a polyethylene glycol wetting agent. The resulting paste is then pulled into a syringe for storage, sterilization, administration, or the like.

Some embodiments provide a kit including a tube pre-filled with a composition described herein, and instructions for use, in a unifying container.

Some embodiments provide a kit including an oral syringe pre-filled with a composition described herein and instruction for use in a unifying container.

Other embodiments provide a kit including a vial or other appropriate container containing a composition described herein and instruction for use in a unifying container.

Still other embodiments provide a kit including a vial or other appropriate container containing a composition described herein, a syringe, and instruction for use in a unifying container.

Some embodiments provide a kit including seven oral syringes pre-filled with a composition described herein and instruction for a week's use in a unifying container.

Other embodiments provide a kit including a vial or other appropriate container containing a composition described herein for seven days of use and instruction for use in a unifying container.

Still other embodiments provide a kit including a vial or other appropriate container containing a composition described herein for seven days of use, seven oral syringes, and instruction for use in a unifying container.

Once housed in a delivery device or included in a kit, the composition/delivery devices/kits can be sterilized using conventional sterilization techniques without substantial degradation to the composition. Without substantial degradation to the composition means that the composition retains greater than 80%, greater than 90%, greater than 95%, or greater than 99% of its activity. In some embodiments, the compositions remain unaffected by sterilization. Sterilization can be by at least one sterilization technique including, but not limited to, gamma irradiation, pressure sterilization, and/or steam sterilization.

In some embodiments, the compositions described herein can retain substantially all potency for at least 14 days. In some embodiments, the compositions described herein can retain substantially all potency for at least 30 days. In some embodiments, the compositions described herein can retain substantially all potency for at least 60 days. In some embodiments, the compositions described herein can retain substantially all potency for at least 90 days. In some embodiments, the compositions described herein can retain substantially all potency for at least 180 days. In some embodiments, the compositions described herein can retain substantially all potency for at least 360 days. In some embodiments, the compositions described herein can retain substantially all potency for longer than 360 days.

Substantially all potency means that the compositions retain at least greater than 80%, greater than 90%, greater than 95%, or greater than 99% of its activity when stored at appropriate conditions. In some embodiments, appropriate conditions can be at room temperature. In some embodiments, appropriate conditions can be without direct light. In some embodiments, appropriate conditions can be under refrigeration or refrigerated.

The compositions described herein can reduce incidence time of a neurological condition. In one embodiment, the compositions described herein can reduce the incidence time of EPM. In some embodiments, the compositions can simply treat EPM.

In some embodiments, the herein described compositions can reduce the incidence time of a neurological condition when compared to treatment with fenbendazole alone by at least about 10%, by at least about 20%, by at least about 50%, by at least about 75%, by at least about 100%, by at least about 150%, by at least about 200%. In some embodiments, treatment time can be reduced by at least 1 week, at least 2 weeks, at least 3 weeks, at least 4 weeks, at least 5 weeks, or at least 6 weeks. In some embodiments, the herein described compositions can fully treat a gastrointestinal condition thereby not requiring indefinite treatment.

As a result of the reduced incidence time, a composition disclosed herein may reduce an unwanted side effect elicited by administration of fenbendazole. In one embodiment, unwanted side effects of fenbendazole include, without limitation, vomiting, drooling, and/or diarrhea.

Further, as a result of the synergistic and/or additive effects of the herein described compositions, a reduced load of drugs may be required to achieve similar results to the drugs alone. In some embodiments, treatment using the described compositions may only require about 10%, about 20%, about 30%, about 40%, about 50%, about 60%, about 70%, or about 80% of a general dose of fenbendazole. In other embodiments, treatment using the described compositions may only require less than 10%, less than 20%, less than 30%, less than 40%, less than 50%, less than 60%, less than 70%, or less than 80% of a general dose of fenbendazole.

In some embodiments, compositions described herein can be administered regularly to aid in preventing neurological conditions. The compositions can be administered daily after treatment to prevent recurrence. In other embodiments, the compositions can be administered before evidence of a neurological condition exists to prevent a neurological condition.

Successful treatment of EPM can comprise an improvement of at least one degree on the modified Mayhew neurological assessment scale:
0=no gait deficits
1=deficits barely perceptible, worsening with head elevation
2=deficits noted at walk
3=deficits noted at rest and walking, nearly falls with head elevation
4=falls/nearly falls at normal gait
5=recumbent

In some embodiments, horses can be successfully treated within about 30 days of treatment. In other embodiments, horses can be successfully treated in less than about 30 days of treatment. In other embodiments, greater than about 80% of horses can be successfully treated within about 30 days of treatment. In other embodiments, greater than about 80% of horses can be successfully treated in less than about 30 days of treatment. In other embodiments, about 80% of horses can be successfully treated after 28 days of treatment.

In other embodiments, about 90% of horses can be successfully treated after 30 days of treatment. In other embodiments, greater than about 90% of horses can be successfully treated within about 30 days of treatment. In other embodiments, greater than about 90% of horses can be successfully treated in less than about 30 days of treatment. In other embodiments, about 90% of horses can be successfully treated after 30 days of treatment.

In other embodiments, about 95% of horses can be successfully treated after 30 days of treatment. In other embodiments, greater than about 95% of horses can be successfully treated within about 30 days of treatment. In other embodiments, greater than about 95% of horses can be successfully treated in less than about 30 days of treatment. In other embodiments, about 95% of horses can be successfully treated after 30 days of treatment.

In some embodiments, horses can be successfully treated within about 15 days of treatment. In other embodiments, horses can be successfully treated in less than about 15 days of treatment. In other embodiments, greater than about 80% of horses can be successfully treated within about 15 days of treatment. In other embodiments, greater than about 80% of horses can be successfully treated in less than about 15 days of treatment. In other embodiments, about 80% of horses can be successfully treated after 14 days of treatment.

In some embodiments, horses can be successfully treated within about 14 days of treatment. In other embodiments, horses can be successfully treated in less than about 14 days of treatment. In other embodiments, greater than about 80% of horses can be successfully treated within about 14 days of treatment. In other embodiments, greater than about 80% of horses can be successfully treated in less than about 14 days of treatment.

In other embodiments, about 90% of horses can be successfully treated after 14 days of treatment. In other embodiments, greater than about 90% of horses can be successfully treated within about 14 days of treatment. In other embodiments, greater than about 90% of horses can be successfully treated in less than about 14 days of treatment. In other embodiments, about 90% of horses can be successfully treated after 14 days of treatment.

In other embodiments, about 95% of horses can be successfully treated after 14 days of treatment. In other embodiments, greater than about 95% of horses can be successfully treated within about 14 days of treatment. In other embodiments, greater than about 95% of horses can be successfully treated in less than about 14 days of treatment. In other embodiments, about 95% of horses can be successfully treated after 14 days of treatment.

In some embodiments, horses can be clinically healed (such that a Western Blot [antibody test] on the horse's cerebrospinal fluid (CSF) shows no evidence of the infection) within about 30 days of treatment. In other embodiments, horses can be clinically healed in less than about 30 days of treatment. In other embodiments, greater than about 80% of horses can be clinically healed within about 30 days of treatment. In other embodiments, greater than about 80% of horses can be clinically healed in less than about 30 days of treatment. In other embodiments, about 80% of horses can be clinically healed after 28 days of treatment.

In other embodiments, about 90% of horses can be clinically healed after 30 days of treatment. In other embodiments, greater than about 90% of horses can be clinically healed within about 30 days of treatment. In other embodiments, greater than about 90% of horses can be clinically healed in less than about 30 days of treatment. In other embodiments, about 90% of horses can be clinically healed after 30 days of treatment.

In other embodiments, about 95% of horses can be clinically healed after 30 days of treatment. In other embodiments, greater than about 95% of horses can be clinically healed within about 30 days of treatment. In other embodiments, greater than about 95% of horses can be clinically healed in less than about 30 days of treatment. In other embodiments, about 95% of horses can be clinically healed after 30 days of treatment.

In some embodiments, horses can be clinically healed within about 15 days of treatment. In other embodiments, horses can be clinically healed in less than about 15 days of treatment. In other embodiments, greater than about 80% of horses can be clinically healed within about 15 days of treatment. In other embodiments, greater than about 80% of horses can be clinically healed in less than about 15 days of treatment. In other embodiments, about 80% of horses can be clinically healed after 14 days of treatment.

In some embodiments, horses can be clinically healed within about 14 days of treatment. In other embodiments, horses can be clinically healed in less than about 14 days of treatment. In other embodiments, greater than about 80% of horses can be clinically healed within about 14 days of treatment. In other embodiments, greater than about 80% of horses can be clinically healed in less than about 14 days of treatment.

In other embodiments, about 90% of horses can be clinically healed after 14 days of treatment. In other embodiments, greater than about 90% of horses can be clinically healed within about 14 days of treatment. In other embodiments, greater than about 90% of horses can be clinically healed in less than about 14 days of treatment. In other embodiments, about 90% of horses can be clinically healed after 14 days of treatment.

In other embodiments, about 95% of horses can be clinically healed after 14 days of treatment. In other embodiments, greater than about 95% of horses can be clinically healed within about 14 days of treatment. In other embodiments, greater than about 95% of horses can be clinically healed in less than about 14 days of treatment. In other embodiments, about 95% of horses can be clinically healed after 14 days of treatment.

In some embodiments, the herein described compositions can be administered without the need for rest or reduced training. In an embodiment of treatment of a race horse, the compositions can be administered during training. In some embodiments, the training can be heavy training in preparation for a race.

In other embodiments, treatment with the herein described compositions can enhance a patient's ability to perform at an optimal level. In some embodiments, prior treatments require a patient to be treated for months, years, or even a lifetime. Unlike the herein described compositions where a patient can train during treatment, prior treatments can prevent training or optimal performance.

In some embodiments, prior treatments require a patient to be treated for months, years, or even a lifetime. Unlike the herein described compositions where a patient can train during treatment, prior treatments can prevent training or optimal performance.

In some embodiments, when administered to a racing horse or camel, treatment with the herein described compositions can enhance the animals performance when compared to being treated using any prior method and/or composition. In some embodiments, the herein described compositions can allow a treated horse or camel to perform at an optimal level within the periods described herein, whereas a horse or camel treated by any prior composition or method can only perform at a diminished level and may never again achieve optimal performance levels. Thus, in some embodiments, the herein described compositions and methods can increase the running performance of a horse or camel when compared to prior treatment compositions and methods.

### Example 1

### Horse Treatment

Two horses suffering from EPM are each treated with a composition including 25 g toltrazuril and 20 g fenbendazole. The composition is stored in a single-use tube. The treatment includes a loading dose of a single tube for each horse on each of day 1, day 3, and day 14. Following the loading dose, a single tube per week per horse is administered

After 30 days of treatment using the composition, both horses are successfully treated.

### Example 2

### Horse Treatment

Two horses suffering from EPM are each treated with a composition including 25 g decoquinate and 20 g fenbendazole. The composition is stored in a single-use tube. The treatment includes a loading dose of a single tube for each horse on each of day 1, day 3, and day 14. Following the loading dose, a single tube per week per horse is administered

After 30 days of treatment using the composition, both horses are completely healed.

### Example 3

### Horse EPM Prevention

A horse is administered a composition once daily including 25 g toltrazuril and 20 g fenbendazole. The horse presents no signs of EPM. The horse never develops EPM.

### Example 4

### Horse EPM Reoccurrence Prevention

A horse is administered daily a composition including 25 g decoquinate and 20 g fenbendazole after being treated for EPM. At the initiation of treatment, the horse presents no signs of EPM. The horse does not develop a reoccurrence of EPM during the year of treatment.

### Example 5

### Scoop Preparation

A scoop is prepared that includes 25 g toltrazuril and 20 g fenbendazole EP powder 100.5, 0.852 g green apple flavor #6005 (sugar free), and 16.52 g of dextrose USP anhydrous powder. The contents are weighed and mixed together one at a time: omeprazole, fenbendazole, apple flavor, and dextrose. The powders are blended until an even color and smooth mixture (free of clumps) is obtained throughout the mixture. Then the mixture is dispensed into jars with a 30 cc scoop.

### Example 6

### Suspension Preparation

A suspension is prepared that includes 250 g toltrazuril, 28.8 mL aqueous green apple flavor (sugar free), 192 g of fenbendazole EP powder 100.5, and 960 mL of polyethylene glycol 300 NF liquid. The contents are weighed and mixed together one at a time: toltrazuril, fenbendazole, and apple flavor. To an appropriate sized beaker is added 50% total volume of the polyethylene glycol. Then, toltrazuril, fenbendazole, and apple flavor are added and then blended. Sodium bicarbonate is added to the mixture and mixed/blended until uniform. The pH is checked.

### Example 7

### Horse Treatment

A 4 year old Thoroughbred Gelding is treated at a barn on the backside of Churchill Downs racetrack in Louisville, Ky. The horse is currently in training. The horse is treated with a composition including 25 g toltrazuril and 20 g fenbendazole each day during the treatment period.

### Example 8

### Horse Treatment

A 4 year old Thoroughbred Colt is treated at a barn on the backside of Churchill Downs racetrack in Louisville, Ky. This horse is currently in training. The horse is treated with a composition including 25 g toltrazuril and 20 g fenbendazole each day during the treatment period. This horse ran (raced) during treatment without adverse effects.

### Example 9

### Suspension Preparation

A suspension is prepared that includes 250 g decoquinate, 28.8 mL aqueous green apple flavor (sugar free), 192 g of fenbendazole EP powder 100.5, and 960 mL of polyethylene glycol 300 NF liquid. The contents are weighed and mixed together one at a time: toltrazuril, fenbendazole, and apple flavor. To an appropriate sized beaker is added 50% total volume of the polyethylene glycol. Then, toltrazuril, fenbendazole, and apple flavor are added and then blended. Sodium bicarbonate is added to the mixture and mixed/blended until uniform. The pH is checked.

### Example 10

### Suspension Preparation

A suspension was prepared by the following method:

1. Obtain beaker large enough to contain PEG 1450. Use beaker that is twice the amount to weigh (If you need 500GM of PEG 1450, use a 1000 mL beaker).

2. Scan and weigh PEG 1450. Melt @ 130° C.-150° C.

3. While the PEG 1450 is melting, obtain mixing vessel large enough to hold final volume.

4. Scan and weigh decoquinate, fenbendazole and apple flavor.

5. Scan PEG 300.

6. Once Step 2 is complete, add PEG 300 to equal 50% total weight to beaker.

7. Add decoquinate, fenbendazole and apple flavor to step 6.

8. Bring to final weight with PEG 300.

9. Blend/Mix until uniform and free of clumps.

10. Package 30 mL in 30 mL Dial-A-Dose syringe

### Example 11

### Scoop Preparation

A scoop is prepared that includes 25 g decoquinate and 20 g fenbendazole EP powder 100.5, 0.852 g green apple flavor #6005 (sugar free), and 16.52 g of dextrose USP anhydrous powder. The contents are weighed and mixed together one at a time: omeprazole, fenbendazole, apple flavor, and dextrose. The powders are blended until an even color and smooth mixture (free of clumps) is obtained throughout the mixture. Then the mixture is dispensed into jars with a 30 cc scoop.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A composition comprising:
at least one antiparasitic drug and at least one antiprotozoal drug;
wherein the composition is formulated as a non-solid for oral administration.

2. The composition of claim 1, wherein at least one of the antiprotozoal drugs comprises decoquinate or toltrazuril.

3. The composition of claim 2, wherein the toltrazuril is present at a 1.25:1 ratio as compared to the antiparasitic drug.

4. The composition of claim 1, wherein at least one of the antiparasitic drugs is fenbendazole.

5. The composition of claim 4, wherein the fenbendazole is present at a 1:1.25 ratio as compared to the antiprotozoal drug.

6. The composition of claim 1 formulated as a liquid.

7. The composition of claim 1 formulated as a paste.

8. The composition of claim 1, further including polyethylene glycol.

9. A composition comprising an antiprotozoal drug and an antiparasitic drug for use in the treatment of a neurological condition in a mammal.

10. The composition of claim 9, wherein the antiprotozoal drug comprises decoquinate or toltrazuril.

11. The composition of claim 10, wherein the toltrazuril is present at a 1.25:1 ratio as compared to the antiparasitic drug.

12. The composition of claim 9, wherein the antiparasitic drug is fenbendazole.

13. The composition of claim 12, wherein the antiparasitic drug is present at a 1:1.25 ratio as compared to the antiprotozoal drug.

14. The composition of claim 9, wherein the composition is administered using an oral syringe.

15. The composition of claim 9, wherein the mammal is a human.

16. The composition of claim 9, wherein the mammal is a horse.

17. The composition of claim 9, wherein the mammal is a camel.

18. The composition of claim 9, wherein said neurological condition is EPM.

19. A method of forming a composition comprising an antiprotozoal drug and an antiparasitic drug, the method comprising:
mixing the drugs in a melted base agent to form the composition.

20. The method of claim 19, wherein the antiprotozoal drug is decoquinate or toltrazuril.

21. The method of claim 20, wherein the toltrazuril is present at a 1.25:1 ratio as compared to the antiparasitic drug.

22. The method of claim 19, wherein the antiparasitic drug is fenbendazole.

23. The method of claim 19, wherein the antiparasitic drug is present at a 1:1.25 ratio as compared to the antiprotozoal drug.

24. The method of claim 19, wherein said composition is formulated as a liquid.

25. The method of claim 19, wherein said composition is formulated as a paste.

26. The method of claim 19, wherein the melted base agent is polyethylene glycol.

27. The method of claim 19, further including a preservative.

28. The method of claim 19, further including a wetting agent.
